(19) United States
(12) Patent Application Publication
(10) Pub. No.: US 2025/0065073 A1
(43) Pub. Date: Feb. 27, 2025

(54) DEVICE FOR DEVELOPING CONCENTRATIONS OF ETERNAL LIFE PRK-1UM OF THREE-MODES

(71) Applicant: Grigorii Petrovich Grabovoi, Belgrade (RS)

(72) Inventor: Grigorii Petrovich Grabovoi, Belgrade (RS)

(21) Appl. No.: 18/945,615

(22) Filed: Nov. 13, 2024

Related U.S. Application Data
(63) Continuation-in-part of application No. 16/504,293, filed on Jul. 7, 2019, now Pat. No. 12,144,599.
(60) Provisional application No. 62/695,756, filed on Jul. 9, 2018.

(51) Int. Cl. A61M 21/00; G09B 19/00

(52) U.S. Cl. CPC A61M 21/00; A61M 2021/0044; A61M 2205/502; A61M 2205/583; A61M 2230/63; G09B 19/00

(57) ABSTRACT

A three-mode device for development of concentration is provided. The device includes a housing, a first optical unit, a second optical unit, a plurality of switches for switching between a plurality of operation modes, and a plurality of lighting units to indicate the plurality of operation modes by emitting a plurality of predetermined light signals. A first lighting unit is configured to emit a first predetermined light signal in the second operation mode and emit a second predetermined light signal in the third operation mode. The device further includes a motion sensor, at least two lasers, and a processing unit. A first laser is configured to operate continuously upon activating the third switch of the plurality of switches. A second laser is in communication with the motion sensor and is configured to switch on upon detection of the motion by the motion sensor.

### CROSS-REFERENCE TO RELATED APPLICATIONS

This application is a Continuation-in-Part of, and claims the priority benefit of, U.S. patent application Ser. No. 16/504,293, filed on Jul. 7, 2019, and entitled “DEVICE OF DEVELOPMENT OF CONCENTRATIONS OF ETERNAL LIFE PRK-1U IS OF THREE-MODES,” which in turn claims priority of U.S. Provisional Patent Application No. 62/695,756, filed on Jul. 9, 2018, and entitled “DEVICE OF DEVELOPMENT OF CONCENTRATIONS OF ETERNAL LIFE PRK-1U IS OF THREE-MODES.” The subject matter of the aforementioned applications is incorporated herein by reference in its entirety.

### TECHNICAL FIELD

This disclosure relates to optical devices. More specifically, this disclosure relates to devices and methods for developing concentration that may be used in a variety of fields including education, training, and therapeutic interventions.

### BACKGROUND

Certain data transmission systems utilize laser-based communication devices that employ laser beams as communication channels between a signal transmitter and a signal receiver. In such systems, a transmission signal is generated by a laser generator in the form of a laser beam, which is modulated based on a data signal by a modulation device connected to a data signal source. The modulated laser beam is then transmitted to the receiver as a receiving signal. The receiving signal is captured by a photodetector and processed by a converting device that converts the modulated laser signal into electrical data signals.

A drawback of these conventional data transmission systems is their low operational reliability, attributed to the complexity of their structure. These systems typically require a large number of complex signal transmitters and receivers, along with a multifunctional communication architecture and a precision guidance system that incorporates moving elements. Furthermore, when transmitting data between a transmitter and a receiver positioned at considerable distances, such as when transmitting data over hundreds or thousands of kilometers via repeaters, the systems experience significant transmission delays, often on the order of tenths of a second. Such delays may be detrimental to data transmission efficiency.

Additionally, these conventional systems exhibit insufficient noise immunity. When obstacles interfere with the laser communication path, signal disruptions or interference in the operation of the systems frequently occur, further reducing the reliability of the data transmission.

### SUMMARY

This summary is provided to introduce a selection of concepts in a simplified form that are further described in the Detailed Description below. This summary is not intended to identify key features or essential features of the claimed subject matter, nor is it intended to be used as an aid in determining the scope of the claimed subject matter.

According to an example embodiment of the present disclosure, a three-mode device for development of concentration is provided. The device may include a housing, a first optical unit attached to the housing, and a second optical unit disposed in the housing and may include a first switch, a second switch, and a third switch. The device may also include a plurality of switches for switching between a plurality of operation modes. The plurality of operation modes may be associated with emittance of a plurality of predetermined light signals. The plurality of operation modes may include at least a first operation mode, a second operation mode, and a third operation mode. The device may further include a plurality of lighting units disposed on the housing and configured to indicate one or more of the plurality of operation modes by emitting one or more of the plurality of predetermined light signals. A first lighting unit of the plurality of lighting units may be configured to emit a first predetermined light signal of the plurality of predetermined light signals in the second operation mode and emit a second predetermined light signal of the plurality of predetermined light signals in the third operation mode. The device may further include a motion sensor disposed on the housing and configured to detect a motion in a predetermined proximity to the motion sensor. The device may further include at least two lasers disposed in the housing. A first laser of the at least two lasers may be configured to operate continuously upon activating the third switch of the plurality of switches. A second laser of the at least two lasers may be in communication with the motion sensor and may be configured to switch on upon detection of the motion by the motion sensor. The device may also include a processing unit disposed in the housing and being in communication with at least the plurality of lighting units, the motion sensor, and the at least two lasers.

According to another embodiment of the present disclosure, a method for development of concentration is provided. The method may include providing a housing, providing a first optical unit attached to the housing, and providing a second optical unit disposed in the housing. The method may further include providing a plurality of switches for switching between a plurality of operation modes associated with emittance of a plurality of predetermined light signals. The plurality of switches may be disposed on the housing and may include a first switch, a second switch, and a third switch. The plurality of operation modes may include at least a first operation mode, a second operation mode, and a third operation mode. The method may further include providing a plurality of lighting units disposed on the housing and configured to indicate one or more of the plurality of operation modes by emitting one or more of the plurality of predetermined light signals. A first lighting unit of the plurality of lighting units may be configured to emit a first predetermined light signal of the plurality of predetermined light signals in the second operation mode and emit a second predetermined light signal of the plurality of predetermined light signals in the third operation mode. The method may further include providing a motion sensor disposed on the housing and configured to detect a motion in a predetermined proximity to the motion sensor. The method may further include providing at least two lasers disposed in the housing. A first laser of the at least two lasers may be configured to switch on in the second operation mode. A first laser of the at least two lasers may be configured to operate continuously upon activating the third switch of the plurality of switches. A second laser of the at least two lasers may be in communication with the motion sensor and may be configured to switch on upon detection of the motion by the motion sensor. The method may further include providing a processing unit disposed in the housing and being in communication with at least the plurality of lighting units, the motion sensor, and the at least two lasers.

Other example embodiments of the disclosure and aspects will become apparent from the following description taken in conjunction with the following drawings.

### BRIEF DESCRIPTION OF DRAWINGS

Exemplary embodiments are illustrated by way of example and not limitation in the figures of the accompanying drawings, in which like references indicate similar elements.

FIG. 1 is a front perspective view of a three-mode device for development of concentration, according to an example embodiment.

FIG. 2 is a top view of a three-mode device for development of concentration, according to an example embodiment.

FIG. 3 is a rear view of a three-mode device for development of concentration, according to an example embodiment.

FIG. 4 is a schematic diagram showing a three-mode device for development of concentration with an opened cover, according to an example embodiment.

FIG. 5 is a schematic diagram showing mutual connection of elements of a three-mode device for development of concentration with respect to each other, according to an example embodiment.

FIG. 6 is an electrical diagram showing the electrical connections of all components of a three-mode device for development of concentration, according to an example embodiment.

FIG. 7 is a flow chart of a method for development of concentration, according to an example embodiment.

FIG. 8 is a flow chart of a computer-implemented method for development of concentration, according to an example embodiment.

FIG. 9 illustrates a computing system that can be used to implement a method for development of concentration, according to an example embodiment.

### DETAILED DESCRIPTION

The following detailed description of embodiments includes references to the accompanying drawings, which form a part of the detailed description. Approaches described in this section are not prior art to the claims and are not admitted to be prior art by inclusion in this section. The drawings show illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical, and operational changes can be made without departing from the scope of what is claimed. The following detailed description is, therefore, not to be taken in a limiting sense, and the scope is defined by the appended claims and their equivalents.

Generally, the embodiments of this disclosure relate to a three-mode device and a method for development of concentration. The device can be used for development of concentration of a user. The device uses an information transmission system that has a high operational reliability, ensures the transmission of information without delay, and is immune to interference. The concentration of the user can be defined as the mental strength of the user to concentrate on a specific goal, such as health, rejuvenation, or eternal life.

The device for development of concentration is based on a principle of similarity. The similarity principle is based on the wave synthesis theory in combination with the unified reality theory. These physical and mathematical theories, experimental results, physical and mathematical calculations, and results of these calculations are described in Grabovoi “Research and Analysis of Fundamental Definitions of Optical Systems in Prevention of Catastrophes and Forecast Oriented Control of Microprocesses,” “Electronic Equipment, Series 3, Microelectronics,” 1999, edition 1 (153). The device and method of the present disclosure are also based on physical principals and structures described in patent No. RU 2148845C1 to Grabovoi titled “Method for preventing catastrophes and equipment for its realization” and patent No. RU2163419C1 to Grabovoi titled “Data transmission system.” Aforementioned documents are included herein by reference in their entirety.

The three-mode device for development of concentration includes a housing and a plurality of components disposed in the housing or attached to the housing. More specifically, the device may include a first optical unit and a second optical unit. The first optical unit may include one or more lenses attached to the housing. A user may be located in proximity to the device. The one or more lenses may be attached to a cover of the housing and may enable the user to affix the user gaze on the one or more lenses. The second optical unit may include a lens disposed in the housing.

The device may further include a plurality of switches for switching between a plurality of operation modes associated with emittance of a plurality of predetermined light signals. The plurality of operation modes may include at least a first operation mode, a second operation mode, and a third operation mode. The device may further include a plurality of lighting units configured to indicate one or more of the plurality of operation modes by emitting one or more of the plurality of predetermined light signals. A first lighting unit of the plurality of lighting units may be configured to emit a first predetermined light signal of the plurality of predetermined light signals in the second operation mode and emit a second predetermined light signal of the plurality of predetermined light signals in the third operation mode.

The device may further include a motion sensor configured to detect a motion in a predetermined proximity to the motion sensor. Specifically, the motion sensor may detect a motion of the user in the predetermined proximity to the motion sensor.

The device may further include at least two lasers disposed in the housing. A first laser of the at least two lasers may be configured to operate continuously upon activating the third switch of the plurality of switches. A second laser of the at least two lasers may be in communication with the motion sensor and may be configured to switch on upon detection of the motion by the motion sensor.

The device may further include a processing unit in communication with at least the plurality of lighting units, the motion sensor, and the at least two lasers. The device may further include a card reader configured to read data from a storage medium. The device may further include a display configured to display the data read by the card reader from the storage medium.

When concentrating, the user may visualize their consciousness as a sphere of sensory elements surrounding their body, supported by the body itself. In the next step, the user may visualize this sphere taking a shape similar to that of the body of the user, and then this sphere in the shape of the body may be absorbed by the surface of each sensory element through the reflection of light emitted by the body. The user can further visualize that the emittance of light from this body-shaped form, upon contacting the surface of this form, extends into the infinite external environment relative to the body of the user. This infinite environment is perceived as eternal reality connected to the user's organism, leading to the development of concentration of the user on eternal life.

According to wave synthesis theory, reality can be conceptualized as the periodic intersection of stationary and dynamic regions, where synthesis of a dynamic wave and a stationary wave occurs at the intersection points. Any phenomenon in reality can be defined as an optical system. Human perception operates via elements of light that contain information. In the case of transmitting information from a user generating the information to be transmitted to a first optical unit, the user can be considered to be a transmitting optical system. The user may generate the information in a form of a biological signal. In an example embodiment, the biological signal is a thought. The information generated as thoughts by the user is received by the first optical unit, on which the user concentrates when generating the thoughts. Since a thought is an electromagnetic wave, the thought can be transmitted as part of an optical system. In an example embodiment, the elements of the first optical unit may be spherical. The spherical shape maximizes the activation of the first optical unit through the internal reflection of biological signals. Biological signals may be generated by electric, electromagnetic, or non-electric fields and can include brainwaves or other signals produced by the human body.

The device of the present disclosure may detect the generation of the biological signals and electromagnetic fields from electromagnetic waves emitted by the user. The device operates on the principle of universal connectivity, with the target of concentration being controlled by artificial intelligence (AI).

The development of concentration is achieved by focusing attention of the user by affixing the user's gaze on one or more lenses, where the one or more lenses act as a receiver of the generated biological signal, and monitoring the results of the concentration. In psychology, it is known that the more a person concentrates, the quicker the goal may be achieved and events may be optimized. In the device of the present disclosure, the electromagnetic fields generated by the biological signal are overlaid with this psychological principle, according to the law of universal connectivity, and goal-oriented concentration control is added. The device enhances the ability to concentrate on creative control.

In the wave synthesis theory, it is known that a thought, which is generated as a radiation, simultaneously possesses two quantum states. The first quantum state is located on the signal transmitter, while the second state is located on the signal receiver. This allows for the creation of a device that ensures development of concentration through interaction with thought processes. A user, as an operator, generates information in the form of radiation of thoughts. To operate the device, the user focuses the thought-generated radiation on lenses located on the cover of the device.

The thought contains the target of concentration. The act of concentration for both the present and future time is performed on the signal transmitter, which consists of lenses. Circular movements of concentration are made from the smaller diameter lenses counterclockwise to the larger diameter lenses.

For concentrations related to past events, the circular movement of thought is performed clockwise, from the smaller diameter lenses to the larger diameter lenses. In this case, the beam of concentration is directed not from above towards the first optical unit, as when focusing on present and future events, but outwards from the second optical unit located inside the device.

The second quantum state of the thought is projected onto the signal receiver, which is configured in the form of the second optical unit located inside the housing of the device.

The principles described above were used for creating the device of the present disclosure that develops concentration by interacting with thoughts. The device universally works for development of concentration for controlling any event (controlling 1), development of concentration for controlling clairvoyance (controlling 2), development of concentration for controlling forecasting (controlling 3), and development of concentration for controlling rejuvenation (controlling 4).

The device for development of concentration includes a first switch. The first switch turns on the device for development of concentration and enables interaction of the device for development of concentration with the electromagnetic field of a user that is located near the working first laser or approaches the device for development of concentration before the second laser is turned on. When the first laser is working and when the second laser is turned on, in the beams of light of increased intensity and radiation density, interactions with the electromagnetic field of the user occur inside the laser beam, which normalize the characteristics of this field, thereby allowing more active development of concentration on events that ensure eternal life and thereby speed up the implementation of these events. This effect is enhanced by the operation of a first lighting unit in a form of a light-emitting diode.

The present disclosure further relates to a computer-implemented method for developing the concentration using the device for development of concentration. The method may include receiving an electrical signal converted by a converter. The electrical signal may be converted from an output signal obtained from the first optical unit based on a biological signal associated with a plurality of electromagnetic fields provided by the user. The electrical signal may be processed by a processing unit using AI to output a signal to be emitted using the second optical unit and one of the lighting units. The signal may be associated with the concentration of the user.

In some example embodiments, the method may include providing one or more lenses as part of a first optical unit for sensing biological signals provided by the user. The method further includes switching between three operation modes using three switches. The method further includes indicating, using one of the lighting units, each of the three operation modes by emitting a predetermined light signal. The method further includes recognizing a biological signal associated with a plurality of electromagnetic fields provided by the user in one of the selected operation modes. The method further includes converting the biological signal into an output signal by processing the signal using the processing unit, generating an output signal, and emitting the output signal using the second optical unit and one of the lighting units.

Referring now to the drawings, various embodiments are described in which like reference numerals represent like parts and assemblies throughout the several views. It should be noted that the reference to various embodiments does not limit the scope of the claims attached hereto. Additionally, any examples outlined in this specification are not intended to be limiting and merely set forth some of the many possible embodiments for the appended claims.

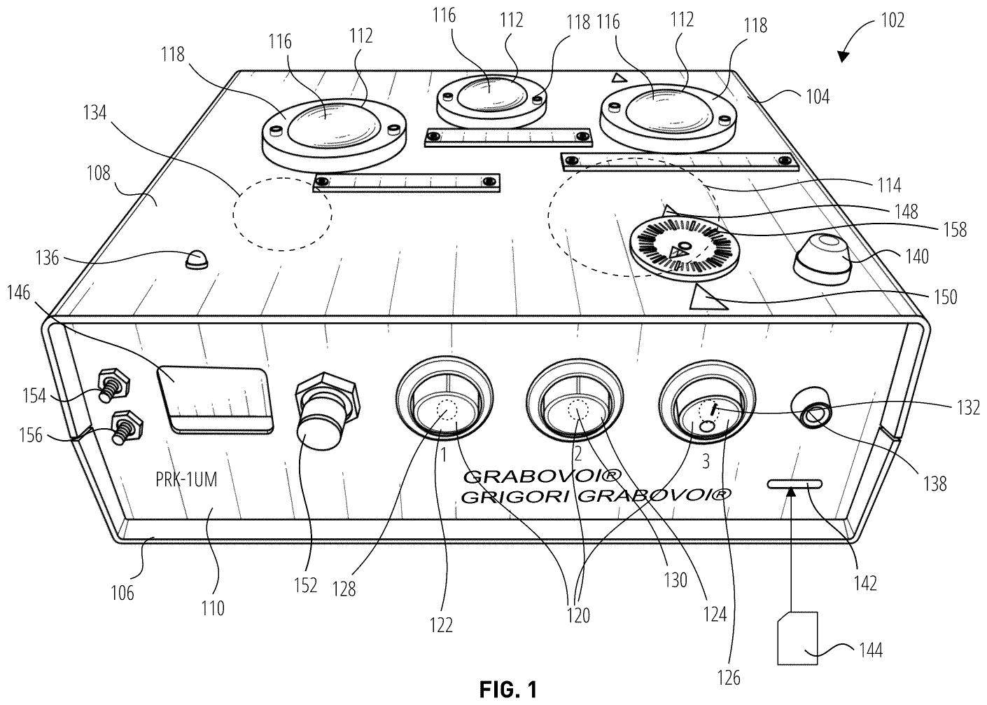

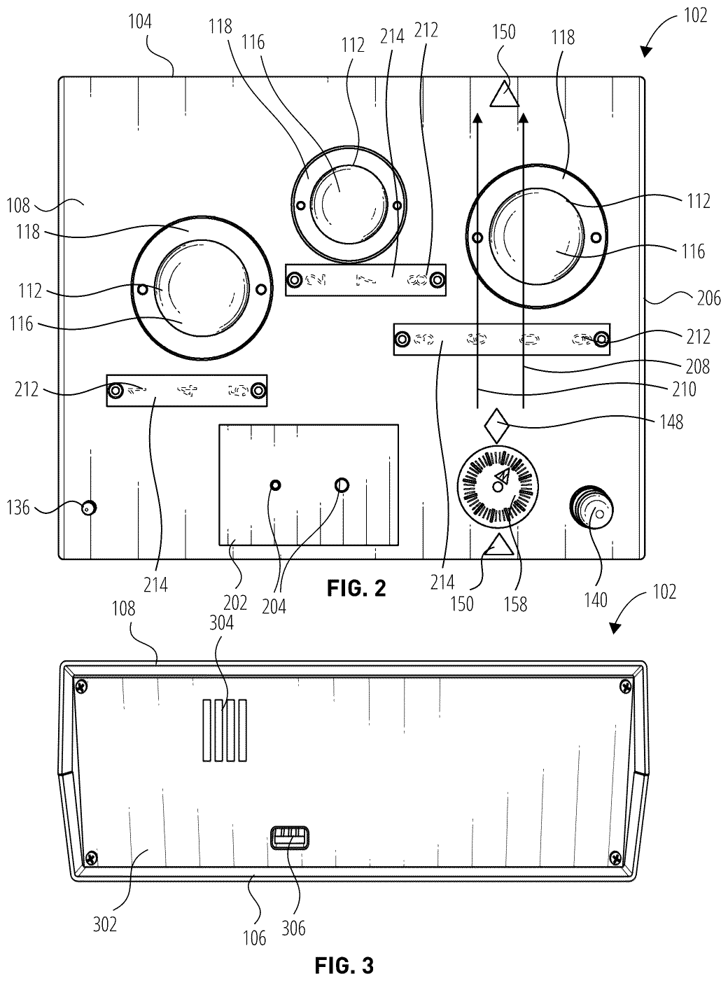

FIG. 1 is a front perspective view of a three-mode device for development of concentration, hereinafter referred to as a device 102, according to an example embodiment. FIG. 2 is a top view of the device 102, according to an example embodiment. FIG. 3 is a rear view of the device 102, according to an example embodiment.

The device 102 may include a housing 104. Referring to FIG. 1, FIG. 2, and FIG. 3, the housing 104 may include a box of a substantially rectangular shape. The housing 104 may include a base 106, a cover 108, a front panel 110, and a back panel 302. The device 102 may further include a first optical unit 112 and a second optical unit 114. The first optical unit 112 may include one or more lenses 116 attached to the housing 104. Specifically, the one or more lenses 116 may be attached to an outer surface of the cover 108. In an example embodiment, the one or more lenses 116 may have a convex shape and may be made of glass. In some example embodiments, each of the one or more lenses 116 may be placed on a plate 118. The plates 118 may be made of glass, metal, or any other applicable material. The one or more lenses 116 may have the same diameter or different diameters. In some example embodiments, the diameter of the one or more lenses 116 may be 20 mm, 25 mm, 60 mm, and any other diameter applicable for a particular embodiment of the device 102. Similarly, the diameter of the plates 118 may be 60 mm, 64 mm, 70 mm, and any other diameter applicable for a particular embodiment of the device 102. Though FIG. 1 shows three lenses 116, example embodiments of the invention may include one lens 116, two lenses 116, and any other number of lenses.

In an example embodiment, the device 102 may include a further plate 202 attached to the housing 104, e.g., to the cover 108 of the housing 104. The device 102 may further include one or more stones 204, such as quartz, diamonds, and any other gemstones. The stone 204 may be disposed on the further plate 202. The stone 204 may be additional optical units. The user may fix the user gaze on the stone 204 when developing the concentration.

The second optical unit 114 may be disposed in the housing 104. In an example embodiment, the second optical unit 114 may include an optical lens having a convex or biconvex shape. The diameter of the second optical unit 114 may be 60 mm or any other applicable diameter. In an example embodiment, the second optical unit 114 may be made of glass.

The device 102 may further include a plurality of switches 120 for switching between a plurality of operation modes associated with emittance of a plurality of predetermined light signals. The plurality of switches 120 may include a first switch 122, a second switch 124, and a third switch 126. The plurality of switches 120 may be disposed on the housing 104, for example, on the front panel 110. The plurality of operation modes may include at least a first operation mode, a second operation mode, and a third operation mode.

The plurality of switches 120 may be provided in a form of up/down switches, which can be set in “up” position for switching on and set in “down” position for switching off. The first switch 122, the second switch 124, and the third switch 126 may be made of a semi-transparent material, such as glass, plastic, and the like. The first switch 122 may have a first light-emitting diode 128, the second switch 124 may have a second light-emitting diode 130, and the third switch 126 may have a third light-emitting diode 132. In an example embodiment, the first switch 122 may be made in red color, the second switch 124 may be made in green color, and the third switch 126 may be made in blue color. When the first light-emitting diode 128 emits light, this light can be seen through the semi-transparent red first switch 122. Similarly, when the second light-emitting diode 130 emits light, this light can be seen through the semi-transparent green second switch 124. Similarly, when the third light-emitting diode 132 emits light, this light can be seen through the semi-transparent blue third switch 126.

The device 102 may further include a plurality of lighting units disposed on the housing 104. The plurality of lighting units may be configured to indicate one or more of the plurality of operation modes by emitting one or more of the plurality of predetermined light signals. The plurality of lighting units may include a first lighting unit 134, a second lighting unit 136, and a third lighting unit 138.

The device 102 may further include a motion sensor 140 disposed on the cover 108 of the housing 104. The motion sensor 140 may be configured to detect a motion of a user in a predetermined proximity to the motion sensor 140. In an example embodiment, the motion sensor 140 may detect the motion within a predetermined distance from the device 102. The predetermined distance may be two meters, thee meters, and any other distance. Other types of sensors for detecting the motion within other distance ranges may be used in some example embodiments.

When all of the switches are turned off, i.e., in a “down” position, the device 102 is switched off. The first switch 122 is used to switch on the device 102. Upon turning on the first switch 122 by putting the first switch 122 into an “up” position, the first operation mode is turned on. The first operation mode is a “universal” operation mode associated with a stationary phase of reality. Upon switching the first switch 122, the first light-emitting diode 128 starts to emit light. As the first switch 122 is red, red light can be seen on the first switch 122 upon the first light-emitting diode 128 emitting the light.

The second switch 124 is used to switch the device into the second operation mode. Upon turning on the second switch 124 by putting the second switch 124 into an “up” position, the second light-emitting diode 130 starts to emit light. As the second switch 124 is green, green light can be seen on the second switch 124 upon the second light-emitting diode 130 emitting the light. In the second operation mode, the stationary phase of reality is enhanced. Upon switching the second switch 124 on and turning the second operation mode on, the first lighting unit 134 located inside the device 102 starts to emit a first predetermined light signal. In an example embodiment, the first predetermined light signal is a static light signal. In an example embodiment, the first lighting unit 134 is a light-emitting diode. The light from the first lighting unit 134 can be seen via the second lighting unit 136, which is a transparent light-emitting diode disposed on the cover 108 and configured to show the light from the first lighting unit 134.

The third operation mode is a pulsed periodic mode in which the dynamic phase of reality is enhanced. To switch the device 102 into the third operation mode, the first switch 122 needs to be turned off and then turned on again, while the second switch 124 needs to stay turned on. In the third operation mode, the first light-emitting diode 128 of the first switch 122 emits a static light signal and the second light-emitting diode 130 of the second switch 124 emits a periodic pulsed light signal (i.e., blinks). Moreover, upon switching on the third operation mode, the first lighting unit 134 located inside the device 102 starts to emit a second predetermined light signal. The second predetermined light signal is a periodic pulsed light signal. The periodic pulsed light signal generated inside the device 102 by the first lighting unit 134 can be seen through the second lighting unit 136, i.e., through the transparent light-emitting diode disposed on the cover 108 and configured to show the light from the first lighting unit 134.

Accordingly, the first lighting unit 134 located inside the device 102 may be configured to emit a first predetermined light signal (a static light signal) in the second operation mode and emit a second predetermined light signal (a periodic pulsed light signal) in the third operation mode.

In order to determine the current operation mode of the device 102, the second switch 124 can be used. If the second light-emitting diode 130 of the second switch 124 is not working, the device 102 operates in the first operation mode. If the second light-emitting diode 130 of the second switch 124 is working by emitting a static light signal, the device 102 operates in the second operation mode, and the static light signal is seen through the second lighting unit 136. If the second light-emitting diode 130 of the second switch 124 is working by emitting a periodic pulsed light signal, the device 102 is in the third operation mode, and the periodic pulsed light signal is seen through the second lighting unit 136.

The device 102 may further include a card reader 142. The card reader 142 may be configured to read data from a storage medium 144. The storage medium 144 may include a secure digital (SD) memory card. The data stored on the storage medium 144 may include numeric series. In an example embodiment, the second predetermined light signal emitted by the third lighting unit 138 in an additional operation mode may be selected based on the numeric series read from the storage medium.

The device 102 may further include a display 146 disposed on the front panel 110 of the housing 104. The display 146 may be configured to display the data read by the card reader 142 from the storage medium 144.

The third switch 126 is used to turn on additional operation modes of the device 102. The third switch 126 may be turned on only in the first operation mode and the second operation mode. The activation of the third operation mode activates the following elements of the device 102: at least two lasers, the motion sensor 140, the card reader 142, the third lighting unit 138 on the front panel 110, and the display 146. Upon turning on the third switch 126, 5 volts of power are supplied to an electrical circuit connecting the elements of the device 102. The light emitted by the at least two lasers can be observed on the back panel 302 of the device 102 through the ventilation slots 304 shown in FIG. 3.

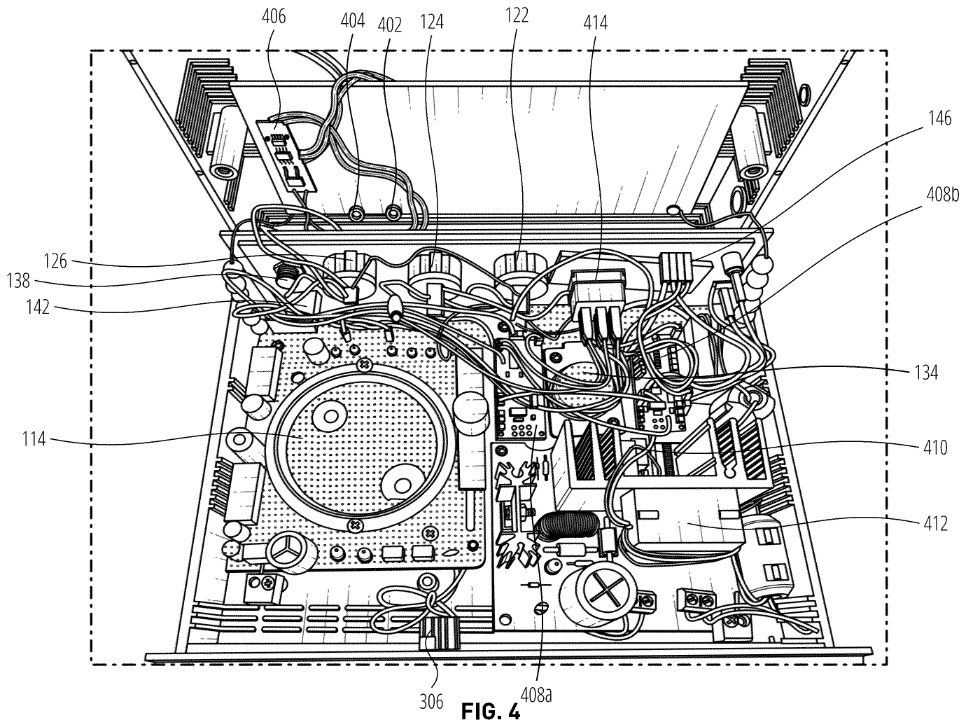

FIG. 4 is a schematic diagram showing the device 102 with an opened cover 108. The device 102 may further include at least two lasers disposed in the housing 104. In an example embodiment, the at least two lasers may be attached to the inner side of the cover 108. As shown in FIG. 4, the at least two lasers may include a first laser 402 and a second laser 404. The second laser 404 may be connected to a sensor plate 406 that activates the motion sensor 140 located on the cover 108.

The first laser 402 may be configured to operate continuously upon activating the third switch 126 of the plurality of switches. Accordingly, the first laser 402 may work the whole time during the operation of the device 102.

The second laser 404 may be in communication with the motion sensor 140 and may be configured to switch on upon detection of the motion by the motion sensor 140.

In an example embodiment, the second optical unit 114 in a form of an optical lens may be configured to reflect light beams produced by the at least two lasers.

The device 102 may further include a processing unit disposed in the housing 104. The processing unit may be in communication with at least the plurality of lighting units, the motion sensor 140, and the at least two lasers.

The at least two lasers may transmit a beam of red light to the second optical unit 114 inside the device 102. The laser beams are directed from the front panel 110 to the back panel 302 strictly parallel to a side plane 206 of the device 102. Indicator 148 and indicator 150 on the cover 108 show the direction of the laser beams. The second laser 404 may be turned on by putting the third switch 126 into an “up” position. The first laser 402 is constantly on upon activation of the additional operation mode. The second laser 404 is connected to the motion sensor 140 and switches on upon detection of the motion by the motion sensor 140. When the device 102 operates in the additional operation mode, in the absence of the user, the second laser 404 turns off 30 seconds after switching on and turns on when the user approaches the device 102 at a predetermined distance (e.g., less than two meters).

The numeric series may be read from the SD card and visualized by using the display 146 or the third lighting unit 138. To activate the operation mode of visualizing the numeric series using the display 146 or the third lighting unit 138, the third switch 126 needs to be tuned off, the SD card needs to be inserted into a slot of the card reader 142, and the third switch 126 needs to be turned on again. Upon activation of this operation mode, text may appear on the display 146 or the third lighting unit 138 may start to blink, i.e., emit a periodic pulsed light signal. To switch between the operation mode of visualizing the numeric series using the display 146 to the operation mode of visualizing the numeric series using the third lighting unit 138, a first button 152 needs to be pressed. Accordingly, the first button 152 is a selection button for selecting one of the operation mode of visualizing the numeric series using the display 146 and the operation mode of visualizing the numeric series using the third lighting unit 138. Upon pressing on the first button 152 in the operation mode of visualizing the numeric series using the display 146, the operation mode of visualizing the numeric series using the third lighting unit 138 is activated and the third lighting unit 138 starts to emit a periodic pulsed light signal with frequency and intensity that corresponds to the number read from the SD card.

The device 102 may further include a plurality of buttons located in proximity to the display 146. The plurality of buttons may enable controlling of an operation of the display 146. The plurality of buttons may include a second button 154 and a third button 156 disposed in proximity to the display 146. The second button 154 and the third button 156 are provided for controlling the information appearing on the display 146. A short press on the second button 154 moves the cursor on the display 146 from the top line down to a file to be opened from the SD card (for example, the filed named “1.TXT” stored on the SD card). A short press on the third button 156 opens the file and the numeric series recorded on the SD card appears on the display 146.

During operation of the continuously operating first laser 402 in the device 102, a static wave of reality is implemented. There are areas of high radiation intensity inside the laser beam with dispersion of the laser beam through the second optical unit 114 into infinite eternal environment. The function of a dynamic wave of reality is provided by the second laser 404, which is switched on by the motion sensor 140.

Focusing on the numeric series read from the SD card when viewed on the display 146 may enable the user to simulate the operation of the third operation mode. Thus, by comparing the operation of the third operation mode and the simulated operation of the third operation mode, the user may be able to accelerate the development of concentration and strengthen the concentration associated with mental models of events.

The third operation mode enables the user to interact with the user's electromagnetic field by being in proximity to the first laser 402 or by approaching the device 102 immediately before the second laser 404 is switched on based on detection of the motion by the motion sensor 140. Upon detection of the motion by the motion sensor 140, the second laser 404 may be turned on. When the first laser 402 is switched on and the second laser 404 is switched on, the interaction with the electromagnetic field of the user occurs in light beams of higher intensity and radiation density inside the laser light beam, which normalizes the characteristics of this electromagnetic field and enables the user to more actively develop concentration on events that are associated with a goal of concentration (such as eternal life) and, therefore, enables the rapid occurrence of these events. This effect is further enhanced by the operation of the first lighting unit 134 and the third lighting unit 138. In an example embodiment, the first lighting unit 134 and the third lighting unit 138 are light-emitting diodes.

In an example embodiment, remote monitoring of the device 102 via the Internet is possible. The impact on the remote users for development of concentration is implemented in the presence of an electromagnetic field due to the radiation of a user's thought and the parameters of the electromagnetic field of the user, which is associated with the electromagnetic field of the Earth on the most distant participants of the electromagnetic field.

In an example embodiment, the device 102 may operate in further operation modes. In a further operation mode, the display 146 may be switched on in an operation mode of reading numeric series. In an example embodiment, the display 146 is selected from an organic light-emitting diode (OLED) display, a liquid crystal display, a light-emitting diode display, and so forth. The display 146 may be switched on by pressing the first button 152 located to the right from the display 146. The numbers and numeric series may be read from a SD-card inserted into the card reader 142 on the front panel 110 of the device 102. The display 146 may display the numeric series read from the SD card. In the operation mode of reading numeric series, the third lighting unit 138 located on the front panel 110 emits periodic pulsed light signals, the frequency and intensity of which corresponds to the figures shown on the display 146.

In accordance with the wave synthesis process, the SD-card implements the transition of the electron into the infinite environment through the periodic pulsed light signals of the third lighting unit 138. This allows for more intensive use of the contact of the matter of consciousness in the area of intersection of the infinite environment with the eternal environment.

The SD card can be read when inserted into the card reader 142. Data contained in the SD card may be transferred to the processing units or microcontrollers. The device 102 has an operation mode that enables the device 102 to read the numeric series from the SD card. In accordance with the wave synthesis process, the numeric series displayed on the display 146 enable the transition of an electron to the infinite eternal environment using the SD card and the instructions running on the processing units. Accordingly, the radiation of a thought transfers data relating to the goal of concentration that corresponds to the numeric series to the infinite eternal environment, in which a system level of goal implementation exists.

Each operation mode of the device 102 can be expanded using the SD card and in view of an operation of the AI. In an example embodiment, the AI may include any type of AI algorithm. Using the numbers on the SD card, the user can perform concentration with the desired control at the required level. Numeric series can be periodically added to the SD card. The numeric series recorded on the SD card are not deleted during factory assembly of the device 102. It is also possible to add individual numeric series or any other pre-selected numeric series. Specific numeric series can ensure the development of concentration for the user. Pressing the first button 152 to the right of the display 146 may open access to a file stored on the SD card. Then, the numbers recorded on the SD card may appear on the display 146.

When the device 102 is in the SD reading mode, the third lighting unit 138 on the right side of the front panel 110 of the device 102 lights up with the light signal and pulsates with a frequency and intensity that corresponds to the numbers read from the SD card.

In order to turn the device 102 off, the first switch 122, the second switch 124, and the third switch 126 need to be turned off.

As shown in FIG. 4, the processing unit of the device 102 may include two processing units shown as a first processing unit 408a and a second processing unit 408b. In an example embodiment, the AI function may be programmed into the processing units. The processing units may be microcontrollers or similar devices for processing and transmitting information contained in the SD card to the display 146 or the third lighting unit 138.

The device 102 may further include a first converter 410 that increases the input voltage from 5 V to the required 9-12 V and further transmits the voltage to a second converter 412. In an example embodiment, the device 102 may have five first converters. The type of the first converter 410 may be MT3608. The processing units may receive electrical signals from the first converter 410 and transmit the electrical signals to a larger second converter 412. The second converter 412 may convert the optical signal emitted by the second optical unit.

In some example embodiments, the device 102 provides the ability to combine three operation modes, thereby creating better concentration. The first switch 122, the second switch 124, and the third switch 126 are connected to a switch 414 for switching signals from the SD card to any of the processing units 408a or 408b. In an example embodiment, the switch 414 may include a 9-pin switch.

Depending on the activity of generation of thoughts by the user and depending on the degree of development of concentration on eternal life in relation to specific events, the AI allows the device 102 to independently turn off the operation modes of the device 102 and then, after a period of time determined by the device 102, to turn on again any of the three operation modes. In an example embodiment, a procedure for activating the AI function of the device 102 was developed. In particular, the first processing unit 408a may process and transmit data read from the SD card to the display 146. The second processing unit 408b may process and transmit data read from the SD card to the third lighting unit 138 in the form of light pulses of varying brightness and duration. The data read from the SD card may include numeric series. Accordingly, the output data of the processing units 408a and 408b that include numeric series for developing concentration can be monitored on the display 146. The brightness and duration of light pulses emitted by the third lighting unit 138 are changed depending on numbers contained in the numeric series. Each number corresponds to its own pulsation frequency and glow strength (brightness). In accordance with the wave synthesis process, the SD card implements the transition of an electron into an infinite environment through periodic pulsed light signals in the third lighting unit 138 due to the dynamics of light scattering in space. Accordingly, the radiation of thought transfers the information on the goal of concentration that corresponds to the numeric series into an infinite eternal environment in which there is a systemic level of implementation of goals.

As shown in FIG. 3, the device 102 may further include a port 306 for connecting to an external power source, such as an electrical grid, a battery, a power bank, and so forth. The port 306 may be disposed on a back panel 302 of the housing 104. In an example embodiment, the port 306 is a universal serial bus (USB)-port. The power provided by the external power source may be 5 V. In some example embodiments, the port 306 may be used for connecting the device 102 to a computing device. In some example embodiments, the device 102 may further include a mini USB port that supplies the power of 5V to the second processing unit 408b when being connected to a connector of the second processing unit 408b.

The device 102 may further include a compass 158 disposed on the cover 108 of the housing 104. Two indicators 148 may be disposed beside the compass 158. The indicators 148 may show direction 208 and direction 210 that are parallel to the directions of light beams produced by the at least two lasers. The compass 158 enables determining the angle between the geographic north-south direction and the direction 208 and direction 210 of the light beams produced by the at least two lasers and shown by the indicators 148. The operation of the device 102 may be started in the initial position of the device 102 when an arrow of the compass 158 is directed at the indicator 148. Further positions of the device 102 may be selected by selecting a direction of the arrow of the compass 158 for each specific user.

The at least two lasers may transmit a beam of red light to the second optical unit 114 located inside the device 102. The beams are directed from the front panel 110 to the back panel 302 strictly parallel to a side plane 206 of the device 102. The lasers may be turned on by pressing the third switch 126. The first laser 402 may be constantly on, and the second laser 404 may be in communication with the motion sensor 140. In the absence of the user, the second laser 404 turns off 30 seconds after turning on and turns on when the user approaches the device 102 at a distance of less than 2 or 3 meters.

The light beam of the first laser 402 acts as a static wave of reality and scatters through the second optical unit 114 into infinite space, i.e., into the eternal environment. The light beam of the second laser 404 acts as the wave function of dynamic reality created inside the device 102, which is activated by the motion sensor 140. The laser beam of the second laser 404 forms a space with the laser beam of the first laser 402 between these laser beams. In this space, an increase in the intensity of radiation of a thought occurs due to the scattering and reflection of laser beams in the second optical unit 114 inside the device 102. When only one light beam is produced, this effect is implemented inside the second optical unit 114 in the reflected and scattered areas of one laser light beam created by the first laser 402.

As shown in FIG. 2, the device 102 may further include a plurality of alphanumeric characters 212 placed on the cover 108 of the housing 104 and one or more plates 214 attached to the cover 108 of the housing 104. The plurality of alphanumeric characters 212 may be placed on the one or more plates 214.

In an example embodiment, the plurality of alphanumeric characters 212 includes a plurality of sets of alphanumeric characters. The alphanumeric characters may include figures, letters, and other alphanumeric characters. A first set of the plurality of sets may include alphanumeric characters of 1, 4, and 5. The first set of alphanumeric characters may be placed near the lens 116 having the largest diameter. A second set of the plurality of sets may include alphanumeric characters of 2, 7, and 8. A third set of the plurality of sets may include alphanumeric characters of 9, 0, 6, and 3. The user may develop the concentration by concentrating on the lenses 116 when fixing the user gaze on the lenses 116 and further concentrating on the alphanumeric characters by fixing the user gaze on some specific alphanumeric characters.

When operating in the third operation mode, each activation of the first lighting unit 134 is carried out by a physical process in the first converter, analogous to a high-impact process. As is well known, an impact action has characteristics of an explosive process. Corresponding physical and mathematical calculations are available that implement the theory of wave synthesis of reality in the initial region where static and dynamic waves of reality intersect. According to the physical and mathematical equations of this theory, physical processes occur in the initial region of the intersection of static and dynamic waves of reality, similar to the well-known Big Bang theory. According to these physical and mathematical equations, the Big Bang theory is a special case of the wave synthesis theory. The Big Bang is a widely accepted cosmological model that describes the early development of the Universe, specifically the start of its expansion from a singular state. According to the Big Bang theory, the further evolution of the Universe depends on an experimentally measurable parameter, namely the average density of matter in the current Universe. If the density does not exceed a certain critical value (known from the theory), the Universe will expand forever. However, if the density exceeds the critical value, the expansion will eventually stop, and the Universe will enter a contraction phase, returning to its original singular state. Current observational data indicate that the average density, within experimental error margins (fractions of a percent), is equal to the critical density.

There are several questions to which the Big Bang theory currently has no answers, but its fundamental principles are supported by reliable experimental data. Modern theoretical physics allows a fairly accurate description of the evolution of matter over time, except for the very early stage lasting during the first hundredth of a second from the “beginning of the Universe.”

In the device of the present disclosure, the impact type of action is explosive, and the physical principle of the evolution of such a system over time is implemented. According to the theory and equations of wave synthesis, this principle includes the initial stage, i.e., the “beginning of the Universe.” Physical and mathematical equations have been developed and practically refined to calculate the characteristics of the explosion based on the measurements of the shockwave parameters.

In scientific studies on protection against continuous or pulsed-periodic laser radiation, powerful laser equipment was used to conduct experiments that confirmed these physical and mathematical equations and calculations. Periodic pulses extrapolated to timescales of 10-2 and 10-17 seconds enabled the calculation of the early-stage development of the Universe—from the “beginning” to approximately one hundredth of a second-using advanced mathematical methods, including complex variable function theory and two other mathematical methods. By applying the wave synthesis theory, a description of the evolution of matter over time from the “beginning of the Universe” was obtained. The propagation of radiation of a thought through the device, along with certain elements of the electromagnetic field and human interaction, in the direction of reverse current flow until disconnection, allows contact with relic radiation, which is part of the physical structure of eternal reality.

The reverse cycle, already diluted by radiation of a human thought and controlled by the electromagnetic field, from the region of cosmic microwave background radiation, allows for the reduction of the average density of matter in the Universe. This contributes to the eternal expansion of the Universe, which is necessary for the eternal life of all. Consequently, this also contributes to the development of concentrations of eternal life, which normalize events in the direction of achieving eternal life for all.

The cyclic operation of the third operation mode enhances consciousness of a human in the fundamental control over reality for eternal life. Through interaction with the relic radiation level, it allows residual processes in electromagnetic and other phenomena to be preserved for an extended period. After switching off the third operation mode and activating the first laser 402 or the second laser 404, these residual processes are intensified. Laser radiation within a monochromatic beam, with many areas of higher density than the neighboring elements of the beam, creates an effect similar to a denser wave. This wave has characteristics analogous to a wave resulting from an explosion. By controlling these explosive characteristics, the physical processes are enhanced, supporting the eternal life of biological organisms in their physical bodies.

In the device 102, the wave-like physical parameters arise from the interaction between the explosion at the electromagnetic field level and monochromatic radiation in the laser beam during the period when residual processes still exist after the third operation mode of the device is triggered. This allows for long-term, continuous development of targeted control, even in isolated or short-term applications.

In the future, the method of the present disclosure may enable the creation of computers based on “delayed” power for each element. By accumulating large quantities of “delayed” energy sources and using them at a single moment to power a controlled, super-powerful pulse, the computing power of the system can be significantly increased. This technology, which ensures the development of concentration associated with eternal life for every individual, provided that consciousness evolves to allow living organisms to ensure the development of concentration associated with eternal life in their physical bodies, is currently a priority.

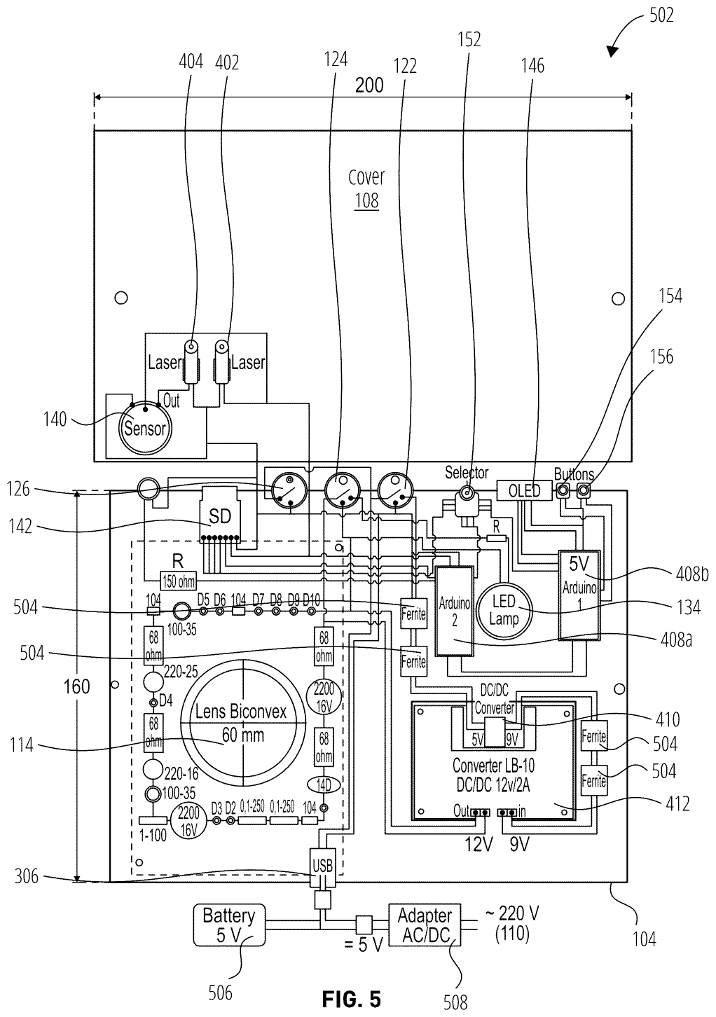

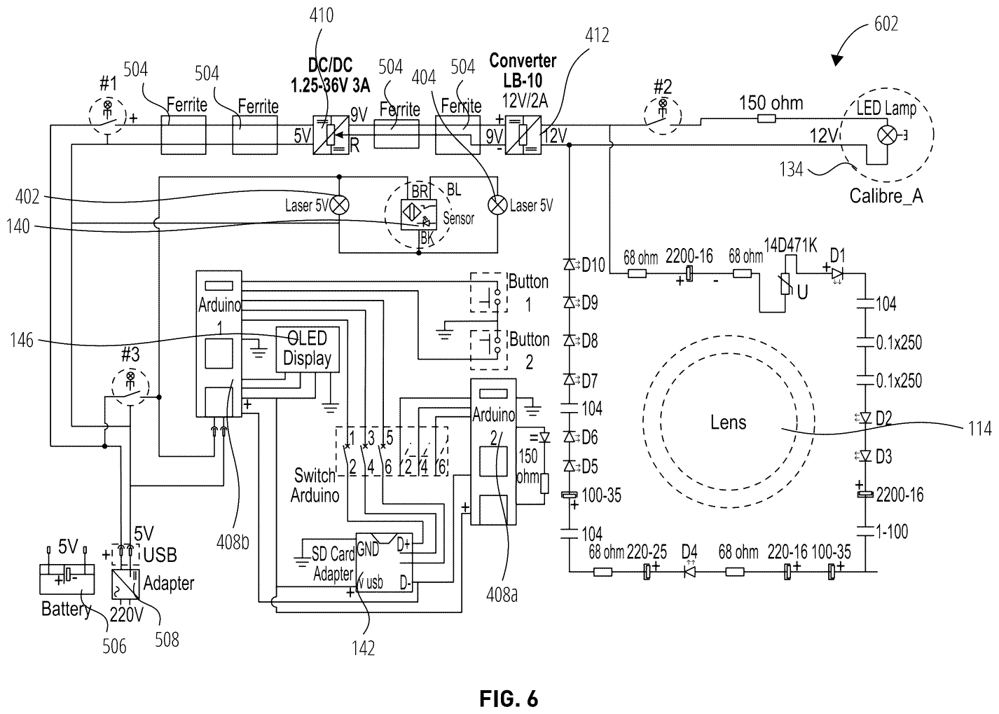

FIG. 5 is a schematic diagram 502 showing mutual connection of elements of the device 102 with respect to each other, according to an example embodiment. FIG. 6 is an electrical diagram 602 showing the electrical connections of all components of the device 102, according to an example embodiment.

As shown in FIG. 5, the type of the first processing units 408a may be Arduino® microcontroller, shown as Arduino 2. The type of the second processing unit 408b also may be Arduino® microcontroller, shown as Arduino 1. In other example embodiments, other types of microcontrollers and microcontroller platforms available for physical computing may be used, such as Parallax Basic Stamp®, Netmedia® BX-24, Phidgets®, MIT® Handyboard, and others offering similar features. All of these tools allow programming in an easy-to-use package. Arduino® microcontrollers also simplify the process of working with microcontrollers and offer teachers, students, and interested hobbyists some advantages over other systems. The data read from the SD card is transmitted to Arduino 1 and Arduino 2 microcontrollers. The switch 414 is used to switch the signal coming from the SD card to Arduino 1 microcontroller or Arduino 2 microcontroller.

In an example embodiment, a USB controller of CH340G type may be used as an interface between a computing device and an Arduino board to transmit program code and commands. In an example embodiment, Arduino 1 and Arduino 2 microcontrollers may include Arduino Pro Mini controllers ATmega168.

The processing units may include Arduino Nano boards, ATmega 168 minicontroller 16 MHz, and CH340G chip (2 pieces), which are software and hardware tools for building systems in the field of electronics and robotics. The software part may consist of a software shell (Integrated Development Environment (IDE)) for writing programs, compiling programs, and programming the hardware. The hardware may be a set of mounted printed circuit boards. The Arduino programming language is C++ with the Wiring® framework.

Arduino 1 microcontroller may be used to process and transmit data contained on the SD card to the display 146. In accordance with the wave synthesis process, the SD card provides transition of an electron into an infinite environment through the static wave of the number on the display 146. Accordingly, the radiation of a thought transfers the information related to the target of concentration and corresponding to the numeric series into an infinite eternal environment in which there is a systemic level of goal implementation.

An Arduino 2 microcontroller may process and transmit data contained on the SD card to the third lighting unit 138 in the form of light pulses of varying brightness and duration. Therefore, the data containing numeric series are transmitted to the third lighting unit 138, which, depending on what numbers are contained on the card, changes the pulsation (i.e., glow) mode. Each number has its own pulsation frequency and glow strength (i.e., brightness). In accordance with the wave synthesis process, the SD card provides the transition of an electron into an infinite environment through periodic pulses of light in the third lighting unit 138 by means of the dynamics of light scattering in a space. Accordingly, the radiation of a thought transfers the information related to the target of concentration and corresponding to the numeric series into an infinite eternal environment in which there is a systemic level of goal implementation.

Using the SD card and instructions for the Arduino NANO board associated with Arduino microcontrollers, the transition of matter into an infinite eternal environment is implemented using a number represented on the display 146 or using the third lighting unit 138. Each operation mode of the device 102 is enhanced by the SD card and using the AI. By using numbers on the SD card, it is possible to develop concentrations with the desired control at the desired level. Numeric series can be periodically added to the SD card. Users can add individual numeric series to the SD card using a computing device. This ensures the development of concentrations for the users and in all selected areas.

In an example embodiment, a plurality of ferrites 504 may be provided in an electric path between the second converter 412 and the first converter 410. The type of the second converter 412 may be an LB-10 converter, which is a direct current (DC)-to-DC converter, 12 V/2A. The type of the first converter 410 may be a DC-to-DC converter, 1.25-36 V 3A.

The device 102 may be connected via the port 306 to an external power source, such as battery 506. The device 102 may be connected via the port 306 to an AC/DC adapter 508.

In an example embodiment, the width of the device 102 may be 160 mm, the length of the device 102 may be 200 mm, and the height of the device 102 may be 65 mm.

The instructions may be recorded to any of Arduino microcontrollers by connecting the device 102 to a computing device and using an “Arduino” computer program by adding a text of the program to a library of the “Arduino” computer program. The Arduino microcontrollers installed on the device 102 may be connected to the computing device by using a USB cable via the port 306. Upon connecting the device 102 to the computing device, a serial port is to be selected. If the Arduino microcontroller is not detected, the “Settings” window should be opened on the computing device to check the status of the port to which the Arduino microcontroller is connected. Then, the “compile” button is pressed. The process of compiling the instructions may be started and may last for about a minute. After the instructions are compiled, their size may be visible in the console. After the process is complete, a light-emitting diode on the Arduino microcontroller may start blinking. This means that the instructions were correctly loaded and launched.

Table 1 below includes instructions for running on the processing units to output numeric series from the SD card to the third lighting unit 138 (LED), according to an example embodiment.

TABLE 1

| Instructions for running on the processing units to output numeric series from | |
|---|---|
| the SD card to the third lighting unit (the light-emitting diode, recited as ‘LED’) | |
| #define FILE_NAME “1.txt” // file to open | |
| #define LED_PIN 7 // pin led is connected to | |
| // functions for every number 0-9 | |
| #define NUM_0_FUNC | blinkLed(2, 500) |
| #define NUM_1_FUNC | blinkLed(3, 250) |
| #define NUM_2_FUNC | pulseLed(2, 100, 500) |
| #define NUM_3_FUNC | pulseLed(3, 10, 300) |
| #define NUM_4_FUNC | pulseLedUp(2, 100, 500) |
| #define NUM_5_FUNC | pulseLedUp(3, 10, 400) |
| #define NUM_6_FUNC | pulseLedUp(4, 10, 300) |
| #define NUM_7_FUNC | pulseLedDown(2, 100, 1000) |
| #define NUM_8_FUNC | pulseLedDown(3, 10, 750) |
| #define NUM_9_FUNC | pulseLedDown(4, 10, 500) |
| /* | |
| * blinkLed(num, delay) - just blinking. Num - number of blinkings/flashes, delay - delay | |
| between blinkings/flashes (ms) | |
| /* | |
| * pulseLed(num, delay, speed) - pulsates (smoothly flares up and fades out) | |
| * pulseLedUp(num, delay, speed) - pulsates upward (flares up smoothly, then goes out | |
| abruptly) | |
| * pulseLedDown(num, delay, speed) - pulsates down (lights up sharply and goes out | |
| smoothly) | |
| * num - number of blinks, delay - delay between blinks (ms), speed - speed at which it | |
| lights up and goes out (the higher the number, the slower) */ | |
| #include <SPI.h> | |
| #include <SD.h> | |
| byte tmp; | |
| bool errorFlag = 1; | |
| void setup( ) { | |
| delay(300); | |
| Serial.begin(9600); | |
| //pinMode(14, OUTPUT); // Used that pin as gnd for testing | |
| pinMode(LED_PIN, OUTPUT); | |
| } | |
| void loop( ) | |
| { | |
| if(errorFlag) // if error happened or boot up | |
| { | |
| Serial.print(“Initializing SD card...”); | |
| if (SD.begin(10)) // try to ini SD card | |
| { | |
| Serial.println(“card initialized.”); | |
| errorFlag = 0; // reset flag is OK, so go into reading loop | |
| } | |
| else | |
| { | |
| Serial.println(“Card failed, or not present”); // else - just wait to try again | |
| delay(3000); | |
| } | |
| } | |
| else | |
| { | |
| File dataFile = SD.open(FILE_NAME); // open file | |
| if (dataFile) // if ok - run reading loop | |
| { | |
| Serial.println(“file opened ”); | |
| Serial.println( ); | |
| while (dataFile.available( )) | |
| { | |
| tmp = dataFile.read( ); | |
| if((tmp>0x2F) & (tmp<0x3A)) // check is character is a 0-9 num | |
| { | |
| tmp -= 0x30; | // get num from an ascii |
| Serial.println(tmp, DEC); | |
| switch(tmp) | // switch between 0-9 |

```
    {
      case 0:
        NUM_0_FUNC;
        break;
      case 1:
        NUM_1_FUNC;
        break;
      case 2:
        NUM_2_FUNC;
        break;
      case 3:
        NUM_3_FUNC;
        break;
      case 4:
        NUM_4_FUNC;
        break;
      case 5:
        NUM_5_FUNC;
        break;
      case 6:
        NUM_6_FUNC;
        break;
          case 7:
            NUM_7_FUNC;
            break;
          case 8:
            NUM_8_FUNC;
            break;
          case 9:
            NUM_9_FUNC;
            break;
        }
      }
    }
    Serial.println(“end of file”);
    Serial.println( );
    dataFile.close( ); // end if file - close it, to open again
  }
  else // if file has filed to be opened - set flag and wait
  {
    Serial.println(“file open error ”);
    Serial.println( );
      errorFlag = 1;
      delay(1000);
    }
  }
}
void blinkLed(byte num, uint16_t del)
{
  for(byte i=0; i<num; i++)
  {
    digitalWrite(LED_PIN, HIGH);
    delay(del);
    digitalWrite(LED_PIN, LOW);
    delay(del);
  }
}
void pulseLed(byte num, uint16_t del, uint16_t spd)
{
  while(num−−)
  {
    for(uint16_t j=1; j<spd; j++)
    {
      digitalWrite(LED_PIN, HIGH);
      delayMicroseconds(j);
      digitalWrite(LED_PIN, LOW);
      delayMicroseconds(spd-j);
    }
    delay(del);
    for(uint16_t j=spd; j; j−−)
    {
      digitalWrite(LED_PIN, HIGH);
      delayMicroseconds(j);
      digitalWrite(LED_PIN, LOW);
      delayMicroseconds(spd-j);
    }
    delay(del);
  }
}
void pulseLedUp(byte num, uint16_t del, uint16_t spd)
{
  while(num−−)
  {
    for(uint16_t j=1; j<spd; j++)
    {
      digitalWrite(LED_PIN, HIGH);
      delayMicroseconds(j);
      digitalWrite(LED_PIN, LOW);
      delayMicroseconds(spd-j);
    }
    delay(del);
  }
}
void pulseLedDown(byte num, uint16_t del, uint16_t spd)
{
  while(num−−)
  {
    for(uint16_t j=spd; j; j−−)
    {
      digitalWrite(LED_PIN, HIGH);
      delayMicroseconds(j);
      digitalWrite(LED_PIN, LOW);
      delayMicroseconds(spd-j);
    }
    delay(del);
  }
}
```

Table 2 below includes instructions for running on the processing units to output numeric series from the SD card to the display, according to an example embodiment.

TABLE 2
Instructions for running on the processing units to output numeric
series from the SD card to the display

```
// Board settings: Clock/Internal 8 MHz, Bootloader/Without bootloader
// Settings
#define ORIENTATION 0 // 0 - right-handed, 1 - left-handed
// pins
#define BTN_UP 3
#define BTN_SET 4
#define BTN_DWN 2
#define OLED_PWR1 5
#define OLED_PWR2 6
#define SD_PWR A1
// libraries
#include “buttonMinim.h”
#include <SPI.h>
#include <SD_fix.h>
#include <GyverOLED.h>
// data and classes
buttonMinim buttUP(BTN_UP);
buttonMinim buttSET(BTN_SET);
buttonMinim buttDWN(BTN_DWN);
GyverOLED oled(0x3C);
Sd2Card card;
SdVolume volume;
SdFile root;
File myFile;
// variables
String filenames = “”;
String setName = “”;
int8_t filesAmount = 0;
byte mode = 0;
int8_t fileSet = 0;
int filePage = 0;
uint32_t btnTimer;
// ================ SETUP ================
void setup( ) {
  pinMode(SD_PWR, OUTPUT);
  pinMode(OLED_PWR1, OUTPUT);
  pinMode(OLED_PWR2, OUTPUT);
  digitalWrite(SD_PWR, 1);
  digitalWrite(OLED_PWR1, 1);
  digitalWrite(OLED_PWR2, 1);
  delay(500);
  oledInit( );
  SDinit( );
  printNames( );
  /*
    // 8 MHz artificially
    CLKPR |= (1 << CLKPCE); // allow prescaler change
    CLKPR = 0x1; // prescaler / 16 (1 MHz)
    SPCR &= ~ (1 << SPR1) | (1 << SPR0); / F_cpu / 4
    SPSR |= (1 << SPI2X); // Double the speed
    TWBR = 0; // max speed TWI
    TWSR &= ~ ((1 << TWPS1) | (1 << TWPS0)); // even faster*/
}
// ================ LOOP ================
void loop( ) {
  if (buttDWN.clicked( )) {
    if (mode == 0) nextFile( );
  else if (mode == 1) nextPage( );
}
if (buttUP.clicked( )) {
  if (mode == 0) prevFile( );
  else if (mode == 1) prevPage( );
}
if (buttDWN.holding( )) {
  if (millis( ) - btnTimer >= 300) {
    btnTimer = millis( );
    if (mode == 0) nextFile( );
    else if (mode == 1) nextPage( );
  }
}
if (buttUP.holding( )) {
  if (millis( ) - btnTimer >= 300) {
    btnTimer = millis( );
    if (mode == 0) prevFile( );
    else if (mode == 1) prevPage( );
  }
}
if (buttSET.clicked( )) {
  if (mode == 0) {
    mode = 1;
    myFile = SD.open(setName, FILE_READ);
    oled.inverse(false);
    printFile( );
  }
}
if (buttSET.holded( )) {
  if (mode == 1) {
      mode = 0;
      myFile.close( );
      printNames( );
    }
  }
}
// ================ CONTROL ================
void nextFile( ) {
  if (++fileSet >= filesAmount) fileSet = filesAmount;
  printNames( );
}
void prevFile( ) {
  if (--fileSet < 0) fileSet = 0;
  printNames( );
}
void nextPage( ) {
  printFile( );
}
void prevPage( ) {
  long pos = myFile.position( ) - 500;
  if (pos < 0) pos = 0;
  myFile.seek(pos);
  printFile( );
}
// ================ PRINT FILE ================
void printFile( ) {
  oled.clear( );
  oled.home( );
  while (!oled.isEnd( )) {
    if((byte)myFile.peek( ) != 255) oled.print((char)myFile.read( ));
    else break;
  }
}
// ================ DISPLAY NAMES ================
void printNames( ) {
  oled.clear( );
  oled.home( );
  int i = 0;
  byte thisPosition = 0;
  byte thisPage = fileSet / 8;
  setName = “”;
  while (true) {
    if (thisPosition == fileSet) {
      oled.inverse(true);
      if (filenames[i] != ‘\n’) setName += filenames[i];
    } else oled.inverse(false);
    if ((thisPosition / 8) >= thisPage) {
      oled.print(filenames[i]);
    }
    if (filenames[i] == ‘\n’) {
      thisPosition++;
      if (thisPosition > filesAmount) break;
    }
    if ((thisPosition / 8) == (thisPage + 1)) break;
    i++;
  }
}
// ================ INITIALIZATION ================
void oledInit( ) {
  Wire.begin( );
  Wire.setClock(400000L);
  oled.init(OLED128x64);
  oled.setContrast(3);
  oled.clear( );
  oled.home( );
  oled.scale2X( );
  oled.println(“SD Reader”);
  oled.scale1X( );
  oled.println(“GRABOVOI ® GRIGORI GRABOVOI ®”);
  oled.println( );
}
void SDinit( ) {
  oled.print(“Init SD...”);
  if (!card.init(SPI_HALF_SPEED, 10)) {
    oled.println(“ error”);
    while (1);
  } else {
    oled.println(“ OK”);
  }
  if (!volume.init(card)) {
    oled.println(“Wrong card!”);
    while (1);
  }
  root.openRoot(volume);
  filenames = root.ls_String(LS_R);
  int i = 0;
  while (true) {
    if (filenames[i] == ‘\n’) filesAmount++;
    if (filenames[i] == ‘\0’) break;
    i++;
  }
  filesAmount−−;
  oled.print((uint32_t)filesAmount);
  oled.println(“ files found”);
  SD.begin(10);
  delay(1000);
  oled.clear( );
  oled.home( );
}
```

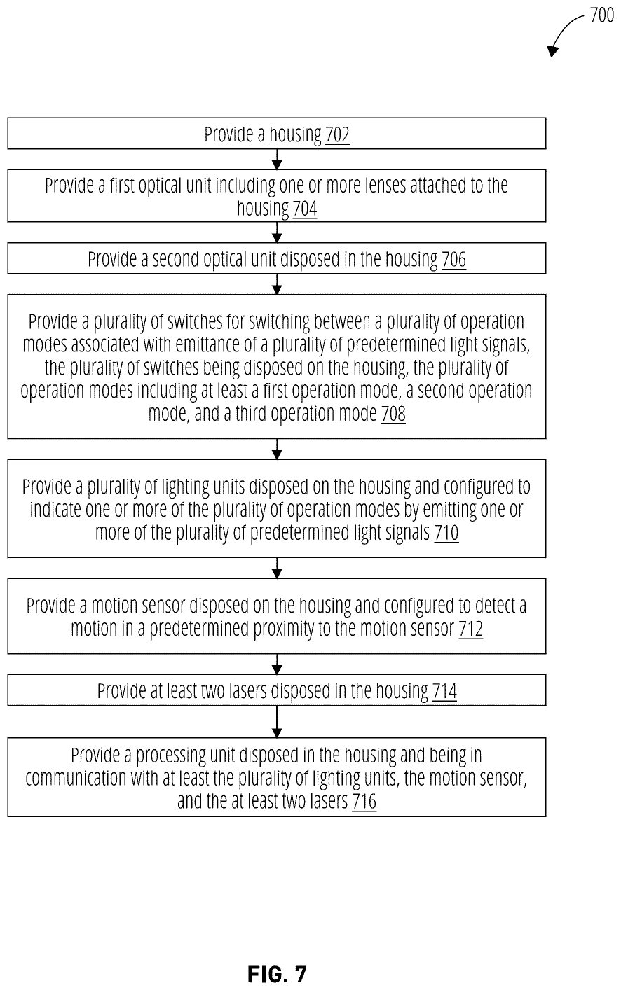

FIG. 7 is a flow chart of a method 700 for development of concentration, according to an example embodiment. In some embodiments, the operations of the method 700 may be combined, performed in parallel, or performed in a different order. Method 700 may also include additional or fewer operations than those illustrated.

The method 700 may commence in block 702 with providing a housing. In block 704, the method 700 may proceed with providing a first optical unit. The first optical unit may include one or more lenses attached to the housing. In block 706, the method 700 may proceed with providing a second optical unit disposed in the housing.

In block 708, the method 700 may include providing a plurality of switches for switching between a plurality of operation modes associated with emittance of a plurality of predetermined light signals. The plurality of switches may be disposed on the housing and may include a first switch, a second switch, and a third switch. The plurality of operation modes may include at least a first operation mode, a second operation mode, and a third operation mode.

In block 710, the method 700 may proceed with providing a plurality of lighting units disposed on the housing and configured to indicate one or more of the plurality of operation modes by emitting one or more of the plurality of predetermined light signals. A first lighting unit of the plurality of lighting units may be configured to emit a first predetermined light signal of the plurality of predetermined light signals in the second operation mode and emit a second predetermined light signal of the plurality of predetermined light signals in the third operation mode.

In block 712, the method 700 may include providing a motion sensor. The motion sensor may be disposed on the housing and configured to detect a motion in a predetermined proximity to the motion sensor.

In block 714, the method 700 may include providing at least two lasers disposed in the housing. A first laser of the at least two lasers may be configured to operate continuously upon activating the third switch of the plurality of switches. A second laser of the at least two lasers may be in communication with the motion sensor and may be configured to switch on upon detection of the motion by the motion sensor.

The second optical unit may include an optical lens. The optical lens may be configured to reflect light beams produced by the at least two lasers.

In block 716, the method 700 may include providing a processing unit. The processing unit may be disposed in the housing and configured to control at least the plurality of lighting units, the motion sensor, and the at least two lasers.

The method 700 may optionally include providing a card reader configured to read data from a storage medium. The method 700 may further include providing a display disposed on the housing. The display may be configured to display the data read by the card reader from the storage medium.

The method 700 may optionally include providing a port for connecting to an external power source. The port may be disposed on the housing.

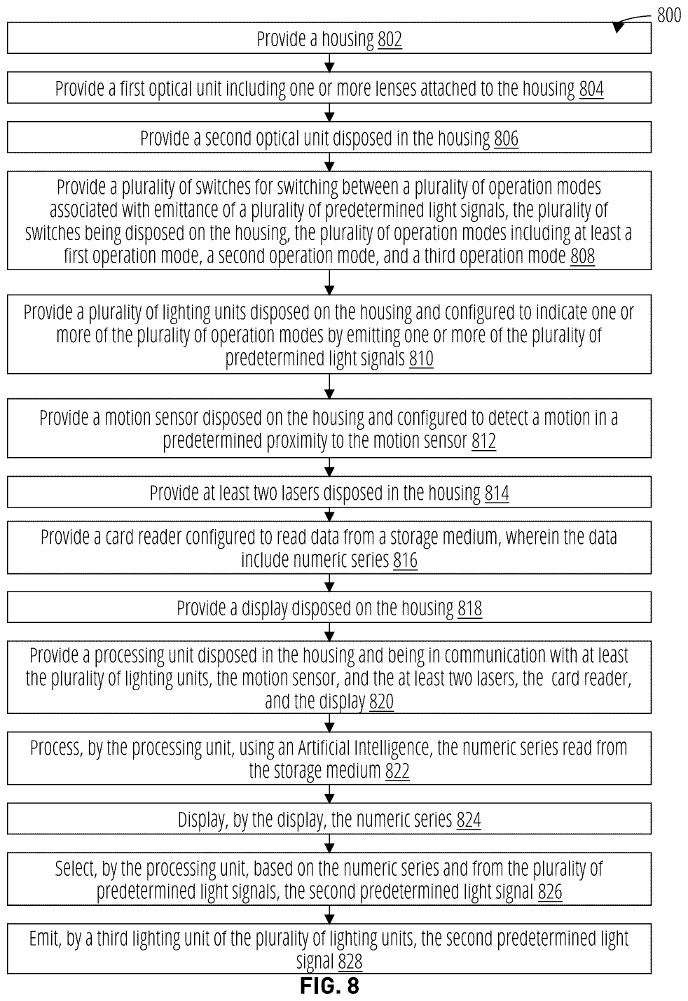

FIG. 8 is a flow chart of a computer-implemented method 800 for development of concentration, according to an example embodiment. In some embodiments, the operations of the method 800 may be combined, performed in parallel, or performed in a different order. The method 800 may also include additional or fewer operations than those illustrated.

The method 800 may commence in block 802 with providing a housing. In block 804, the method 800 may proceed with providing a first optical unit. The first optical unit may include one or more lenses attached to the housing. In block 806, the method 800 may include providing a second optical unit disposed in the housing.

In block 808, the method 800 may include providing a plurality of switches for switching between a plurality of operation modes associated with emittance of a plurality of predetermined light signals. The plurality of switches may be disposed on the housing and may include a first switch, a second switch, and a third switch. The plurality of operation modes may include at least a first operation mode, a second operation mode, and a third operation mode.

In block 810, the method 800 may include providing a plurality of lighting units. The plurality of lighting units may be disposed on the housing and configured to indicate one or more of the plurality of operation modes by emitting one or more of the plurality of predetermined light signals. A first lighting unit of the plurality of lighting units may be configured to emit a first predetermined light signal of the plurality of predetermined light signals in the second operation mode and emit a second predetermined light signal of the plurality of predetermined light signals in the third operation mode.

In block 812, the method 800 may proceed with providing a motion sensor. The motion sensor may be disposed on the housing and configured to detect a motion in a predetermined proximity to the motion sensor.

In block 814, the method 800 may include providing at least two lasers disposed in the housing. A first laser of the at least two lasers may be configured to operate continuously upon activating the third switch of the plurality of switches. A second laser of the at least two lasers may be in communication with the motion sensor and may be configured to switch on upon detection of the motion by the motion sensor.

In block 816, the method 800 may proceed with providing a card reader configured to read data from a storage medium. The data may include numeric series. In block 818, the method 800 may include providing a display disposed on the housing. In block 820, the method 800 may include providing a processing unit disposed in the housing and being in communication with at least the plurality of lighting units, the motion sensor, and the at least two lasers, the card reader, and the display.

In block 822, the method 800 may proceed with processing, by the processing unit using AI, the numeric series read from the storage medium. In block 824, the method 800 may include displaying, by the display, the numeric series. In block 826, the method 800 may include selecting, by the processing unit, the second predetermined light signal from the plurality of predetermined light signals. The processing unit may select the second predetermined light signal based on the numeric series. In block 828, the method 800 may include emitting, by the third lighting unit, the second predetermined light signal.

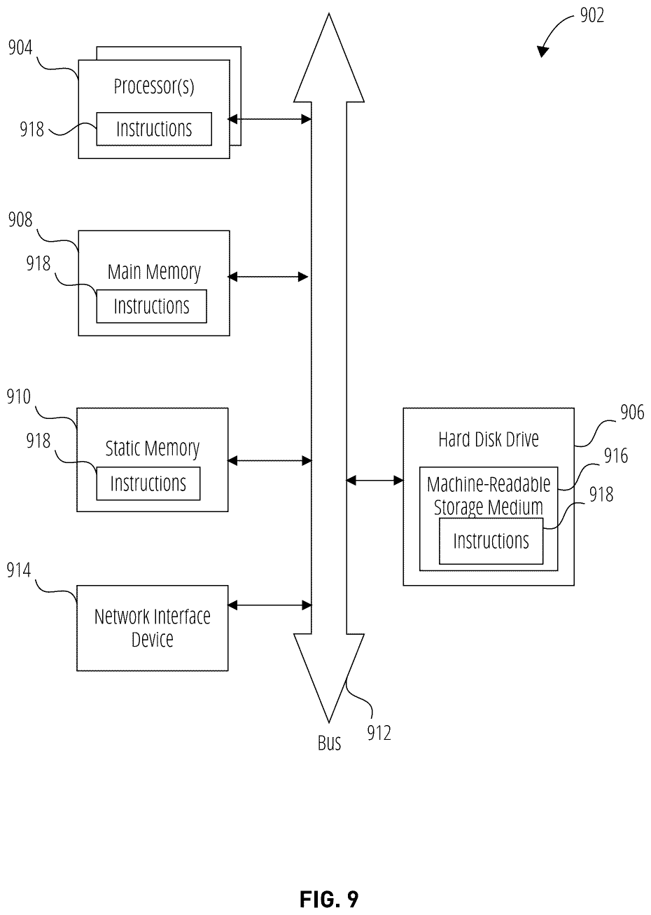

FIG. 9 shows a diagrammatic representation of a computing device for a machine in the exemplary electronic form of a computer system 902, within which a set of instructions for causing the machine to perform any one or more of the methodologies discussed herein can be executed. In various exemplary embodiments, the machine operates as a standalone device or can be connected (e.g., networked) to other machines. In a networked deployment, the machine can operate in the capacity of a server or a client machine in a server-client network environment, or as a peer-to-peer (or distributed) network environment. The machine can be a personal computer (PC), a tablet PC, a set-top box, a cellular telephone, a digital camera, a portable music player (e.g., a portable hard drive audio device, such as a Moving Picture Experts Group Audio Layer 3 (MP3) player), a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 902 may include a processor or multiple processor(s) 904, a hard disk drive 906, a main memory 908, and a static memory 910, which communicate with each other via a bus 912. The computer system 902 may also include a network interface device 914. The hard disk drive 906 may include a machine-readable storage medium 916, which stores one or more sets of instructions 918 embodying or utilized by any one or more of the methodologies or functions described herein. The instructions 918 can also reside, completely or at least partially, within the main memory 908, the static memory 910, and/or within the processor(s) 904 during execution thereof by the computer system 902. The main memory 908, the static memory 910, and the processor(s) 904 may also constitute machine-readable media.

While the machine-readable storage medium 916 is shown in an exemplary embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “machine-readable medium” shall accordingly be taken to include, but not be limited to, solid-state memories, optical and magnetic media. Such media can also include, without limitation, hard disks, floppy disks, NAND or NOR flash memory, digital video disks, Random Access Memory, Read-Only Memory, and the like.

The example embodiments described herein may be implemented in an operating environment comprising software installed on a computer, in hardware, or in a combination of software and hardware.

Thus, a three-mode device for development of concentration and a method for development of concentration have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A three-mode device for development of concentration, the device comprising:
a housing;
a first optical unit including one or more lenses attached to the housing;
a second optical unit disposed in the housing;
a plurality of switches for switching between a plurality of operation modes associated with emittance of a plurality of predetermined light signals, the plurality of switches being disposed on the housing and including a first switch, a second switch, and a third switch, the plurality of operation modes including at least a first operation mode, a second operation mode, and a third operation mode;
a plurality of lighting units configured to indicate one or more of the plurality of operation modes by emitting one or more of the plurality of predetermined light signals, wherein a first lighting unit of the plurality of lighting units is configured to:
emit a first predetermined light signal of the plurality of predetermined light signals in the second operation mode; and
emit a second predetermined light signal of the plurality of predetermined light signals in the third operation mode;
a motion sensor disposed on the housing and configured to detect a motion in a predetermined proximity to the motion sensor;
at least two lasers disposed in the housing, wherein:
a first laser of the at least two lasers is configured to operate continuously upon activating the third switch of the plurality of switches; and
a second laser of the at least two lasers is in communication with the motion sensor and is configured to switch on upon detection of the motion by the motion sensor; and
a processing unit disposed in the housing and being in communication with at least the plurality of lighting units, the motion sensor, and the at least two lasers.

2. The device of claim 1, further comprising a card reader configured to read data from a storage medium.

3. The device of claim 2, further comprising a display disposed on the housing, wherein the display is configured to display the data read by the card reader from the storage medium.

4. The device of claim 2, wherein:
the storage medium includes a secure digital (SD) memory card; and
the data include numeric series.

5. The device of claim 4, wherein the second predetermined light signal emitted by a third lighting unit of the plurality of lighting units in the third operation mode is selected based on the numeric series read from the storage medium.

6. The device of claim 3, further comprising a plurality of buttons located in proximity to the display, wherein the plurality of buttons enable controlling of an operation the display.

7. The device of claim 1, further comprising:
a compass disposed on the housing; and
two indicators disposed beside the compass, the two indicators showing a direction of light beams produced by the at least two lasers.

8. The device of claim 1, further comprising:
a plurality of alphanumeric characters placed on the housing; and
one or more plates attached to the housing, wherein the plurality of alphanumeric characters are placed on the one or more plates.

9. The device of claim 8, wherein:
the plurality of alphanumeric characters includes a plurality of sets of alphanumeric characters;
a first set of the plurality of sets includes alphanumeric characters of 1, 4, and 5;
a second set of the plurality of sets includes alphanumeric characters of 2, 7, and 8; and
a third set of the plurality of sets includes alphanumeric characters of 9, 0, 6, and 3.

10. The device of claim 1, wherein the second optical unit includes an optical lens, the optical lens being configured to reflect light beams produced by the at least two lasers.

11. The device of claim 1, wherein a third lighting unit of the plurality of lighting units is configured to emit a third predetermined light signal of the plurality of predetermined light signals.

12. The device of claim 1, wherein:
the housing includes a cover; and
the one or more lenses are attached to the cover.

13. The device of claim 1, further comprising a port for connecting to an external power source, the port being disposed on the housing.

14. The device of claim 1, wherein the first predetermined light signal is a static light signal and the second predetermined light signal is a periodic pulsed light signal.

15. A method for development of concentration, the method comprising:
providing a housing;
providing a first optical unit including one or more lenses attached to the housing;
providing a second optical unit disposed in the housing;
providing a plurality of switches for switching between a plurality of operation modes associated with emittance of a plurality of predetermined light signals, the plurality of switches being disposed on the housing and including a first switch, a second switch, and a third switch, the plurality of operation modes including at least a first operation mode, a second operation mode, and a third operation mode;
providing a plurality of lighting units configured to indicate one or more of the plurality of operation modes by emitting one or more of the plurality of predetermined light signals, wherein a first lighting unit of the plurality of lighting units is configured to:
emit a first predetermined light signal of the plurality of predetermined light signals in the second operation mode; and
emit a second predetermined light signal of the plurality of predetermined light signals in the third operation mode;
providing a motion sensor disposed on the housing and configured to detect a motion in a predetermined proximity to the motion sensor;
at least two lasers disposed in the housing, wherein:
a first laser of the at least two lasers is configured to operate continuously upon activating the third switch of the plurality of switches; and
a second laser of the at least two lasers is in communication with the motion sensor and is configured to switch on upon detection of the motion by the motion sensor; and
providing a processing unit disposed in the housing and being in communication with at least the plurality of lighting units, the motion sensor, and the at least two lasers.

16. The method of claim 15, further comprising providing a card reader configured to read data from a storage medium.

17. The method of claim 16, further comprising providing a display disposed on the housing, wherein the display is configured to display the data read by the card reader from the storage medium.

18. The method of claim 15, further comprising providing a port for connecting to an external power source, the port being disposed on the housing.

19. The method of claim 15, wherein the second optical unit includes an optical lens, the optical lens being configured to reflect light beams produced by the at least two lasers.

20. A computer-implemented method for development of concentration, the method comprising:
providing a housing;
providing a first optical unit including one or more lenses attached to the housing;
providing a second optical unit disposed in the housing;
providing a plurality of switches for switching between a plurality of operation modes associated with emittance of a plurality of predetermined light signals, the plurality of switches being disposed on the housing and including a first switch, a second switch, and a third switch, the plurality of operation modes including at least a first operation mode, a second operation mode, and a third operation mode;
providing a plurality of lighting units configured to indicate one or more of the plurality of operation modes by emitting one or more of the plurality of predetermined light signals, wherein a first lighting unit of the plurality of lighting units is configured to:
emit a first predetermined light signal of the plurality of predetermined light signals in the second operation mode; and
emit a second predetermined light signal of the plurality of predetermined light signals in the third operation mode;
providing a motion sensor disposed on the housing and configured to detect a motion in a predetermined proximity to the motion sensor;
at least two lasers disposed in the housing, wherein:
a first laser of the at least two lasers is configured to operate continuously upon activating the third switch of the plurality of switches; and
a second laser of the at least two lasers is in communication with the motion sensor and is configured to switch on upon detection of the motion by the motion sensor;
providing a card reader configured to read data from a storage medium, wherein the data include numeric series;
providing a display disposed on the housing;
providing a processing unit disposed in the housing and being in communication with at least the plurality of lighting units, the motion sensor, and the at least two lasers, the card reader, and the display;
processing, by the processing unit, using, an Artificial Intelligence, the numeric series read from the storage medium;
displaying, by the display, the numeric series;
selecting, by the processing unit, based on the numeric series and from the plurality of predetermined light signals, the second predetermined light signal; and
emitting, by a third lighting unit of the plurality of lighting units, the second predetermined light signal.
